(19) United States
(12) Patent Application Publication
(10) Pub. No.: US 2006/0084105 A1
(43) Pub. Date: Apr. 20, 2006

(54) Arrays for detection of products of mRNA splicing

(76) Inventors: Manuel Ares, Santa Cruz, CA (US); Tyson Andrew Clark, Santa Cruz, CA (US); Charles Walsh Sugnet, Santa Cruz, CA (US); Valerie Ann Welch, Santa Cruz, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/271,536

(22) Filed: Nov. 9, 2005

Related U.S. Application Data
(62) Division of application No. 10/423,802, filed on Apr. 25, 2003.
(60) Provisional application No. 60/377,870, filed on May 2, 2002.

(51) Int. Cl. C12Q 1/68; C07H 21/04

(52) U.S. Cl. 435/6; 536/24.3

(57) ABSTRACT

The invention features an array comprising at least one set of nucleic acid probes for detection of gene products that are produced by mRNA splicing of a selected gene, wherein each probe set is specific for a selected gene, and wherein the probe set minimally comprises a splice junction probe and either an intron probe or an exon probe. The splice junction probe hybridizes specifically to a sequence corresponding to a preselected, non-genomic sequence present in a product of mRNA splicing, while the intron probe hybridizes specifically to a sequence corresponding to an intronic sequence present in unspliced mRNA. The exon probe hybridizes specifically to a sequence corresponding to an exonic sequence of the gene. The intron probe and the exon probe may each serve as an internal control. The invention also features methods of using the array to analyze mRNA splice products in a sample.

### CROSS-REFERENCE TO RELATED APPLICATIONS

This application claims the benefit of U.S. provisional application Ser. No. 60/377,870, filed May 2, 2002, which is incorporated herein by reference in its entirety.

### GOVERNMENT RIGHTS

This invention was made with government support under federal grant no. GM40478 awarded by the National Institutes of Health. The United States Government may have certain rights in this invention.

### FIELD OF THE INVENTION

The invention relates to use of nucleic acid probes in the analysis of nucleic acid corresponding to gene products, particularly to analysis of gene products that result from mRNA splicing.

### BACKGROUND OF THE INVENTION

Most eukaryotic protein-coding genes contain exons which are interrupted by introns. Genes are transcribed into a large RNA molecule (“pre-mRNA”) containing both introns and exons. The splicing apparatus generates from the pre-mRNA various mRNA isoforms—or “mRNA spliced variants”—by combining different exons into the mRNA transcript. The spliceosome thus acts on transcripts of the eukaryotic genome to create sequences not found in genomic DNA (J. P. Staley, C. Guthrie, Cell 92, 315-26 (1998)). By its nature and position in the gene expression pathway, splicing expands the possible interpretations of genomic information, and does so under developmental and environmental influence (D. L. Black, Cell 103, 367-70. (2000)).

Alternative splicing arises when the splicing machinery varies what it recognizes as introns and exons. The types of alternative splicing include usage of alternative 5′ or 3′ splice sites, exon skipping, intron retention and mutual exclusion of exons. In about 30-50% of human genes, multiple mRNAs with distinct protein-coding potential can be created from a single gene. Several simple examples occur in genes implicated in cancer and apoptosis. For example the bcl-x gene is involved in apoptosis, and produces two protein isoforms with distinct functions by alternative splicing. Bcl-xS protein promotes apoptosis, while bcl-xL suppresses apoptosis. This binary example belies the complexity that alternative splicing can generate. Consider a gene with multiple tandem cassette exons, any one of which can be included or skipped. The number of mRNA isoforms that could be produced equals 2ⁿ, where n is the number of cassette exons. Therefore, a gene such as CD44 with 9 cassette exons and two alternative C-terminal coding exons can produce 1024 possible mRNA isoforms. Since the CD44 protein could exist in many forms, a broad spectrum of subtly different CD44 activities in cell adhesion are probable. Developing parallel assays that can distinguish between the mRNA isoforms that generate these different proteins will be critical to understanding the roles of the different protein isoforms.

Some arrays for analysis of alternative splicing are provided in the art. Hu et al (Genome Research 11:1237-1245, 2001) disclose the use of DNA microarrays for the purpose of detecting alternative splicing in different rat tissues. Their technology relies on sequence information derived from comparing mature mRNAs only, and does not require knowledge of exon-exon splice junctions nor any intronic or other genomic sequence. Each gene of the microarray is represented by a set of twenty pairs of 25-mer oligonucleotides designed from EST and cDNA sequence information. Alternative splice variants are detected by virtue of the loss of hybridization signal from one or more of the probes in one tissue type versus another. Thus, the method detects alternative splicing only by inference. Furthermore, since the method does not measure formation of the particular splice junction itself, it can only be effective at detecting the predominant mRNA isoform in a given tissue sample.

Shoemaker et al (Nature 409: 922-927, 2001) discloses a method for experimentally confirming the existence of exons predicted by bioinformatics algorithms, then refining knowledge of the structure of the confirmed exons. The method involves construction and sequential use of two types of DNA microarrays. The first array comprises oligonucleotide probes of predicted exons. This ‘exon-array’ is used to experimentally confirm exons predicted from bioinformatics algorithms. Hybridization of a given probe to mRNA from a particular tissue type indicates that the exon is ‘authentic’. Exons are grouped into genes based on observations of coordinated expression of adjacent exons in a variety of tissues.

After determining the actual presence of a predicted exon in an mRNA sample, Shoemaker et al. disclose that the region of the genomic sequence containing the exon is then fine structure mapped using a ‘tiling array’. Tiling arrays are constructed with overlapping oligonucleotides which blanket the sequence of the genomic region of interest. Tiling arrays delimit the endpoints of the exons and are effective at estimating the location of intron-exon junctions in genomic DNA to within 20-30 bp. The technique presented by Shoemaker et al. is useful primarily for determining the existence and approximate structure of predicted exons, thereby providing an experimental method for annotation of genome sequences. However, the method is limited to the detection of the predominant mRNA isoform in each tissue type, since it relies on sequence information from one source only, in this case the genomic DNA.

PCT publication no. WO 01/57252: “Methods and Apparatus for High-Throughput Detection and Characterization of Alternatively Spliced Genes” discloses a “single exon microarray” for experimentally confirming exons predicted from genomic sequence data using bioinformatics algorithms. This method is similar to the Shoemaker et al reference discussed above. Oligonucleotide probes that make up the single exon microarray are comprised of predicted exonic sequences derived from genomic DNA. The array is hybridized with mRNA from different tissues, and based on the intensity of the hybridization signal of adjacent exons, conclusions are drawn about the different RNA isoforms present in different tissues. Identification of spliced and unspliced transcripts is made inferentially by comparison of fluorescence intensities of adjacent probes in different tissues.

As such, there is a need in the field for tool that allows for detection of products of mRNA splicing on a single array. The present invention addresses this need.

### SUMMARY OF THE INVENTION

The invention features an array comprising sets of nucleic acid probes for detection of gene products that are produced by mRNA splicing of a selected gene, wherein each probe set is specific for a selected gene, and wherein the probe set minimally comprises a splice junction probe and either an intron probe or an exon probe. The splice junction probe hybridizes selectively to a sequence corresponding to a pre-selected, non-genomic sequence present in a product of mRNA splicing, whereas the exon probe hybridizes selectively to a sequence corresponding to an exonic sequence of the gene and the intron probe hybridizes selectively to a sequence corresponding to an intronic sequence present in unspliced mRNA. Either the exon or intron probe may serve as an internal control. The invention also features methods of using the array to analyze mRNA splice products in a nucleic sample.

One advantage of the invention is that analysis of many possible RNA splice product for several genes can be accomplished using a single array and in a single step.

Another advantage of the invention is that the data generated using the arrays and methods of the invention can be used to assess the frequency of splicing of a selected gene.

These and other advantages, and features of the invention will become apparent to those persons skilled in the art upon reading the details of the invention as more fully described below.

### BRIEF DESCRIPTION OF THE FIGURES

The patent or application file contains at least one drawing executed in color. Copies of this patent or patent application publication with color drawings will be provided by the office upon request and payment of the necessary fee.

FIG. 1A is a schematic showing a design of a nucleic acid array of the invention. Arrays in this embodiment comprise three oligonucleotide probes for each intron-containing gene, as well as probes for control intronless genes. Intron probes (red) detect unspliced RNA and lariats. Splice junction probes (green) detect spliced mRNA. Exon probes (blue) detect both spliced and unspliced RNAs. Data is normalized to intronless genes (yellow).

FIG. 1B is a set of scatter plots of probe intensities during heat shift of prp4-1. Raw intensity (log₁₀ scale) of each spot without background subtraction or normalization is shown for wt (Cy3, x-axis) and mutant cells (Cy5, y-axis), color-coded for probe type as in FIG. 1A.

FIG. 1C is a set of scatter plots of probe intensities for deletion mutants. Data plotted as in FIG. 1B.

FIGS. 2A-B are illustrations of hierarchical clustering of Splice Junction (SJ) and Intron Accumulation (IFN-α) Indexes.

FIG. 2A is a schematic providing a comparison of the Clusters. Length of tree branches are inversely related to correlation coefficients of joined nodes. Shaded boxes highlight genes that are known to function together.

FIG. 2B is an exemplary SJ Index Cluster. The deletion mutants are clustered on the horizontal axis with intron-containing genes on the vertical axis. Green squares represent a decrease in SJ index.

FIGS. 3A-3B summarize experimental results showing RT-PCR validation of microarray data.

FIG. 3A is a set of photographs illustrating RT-PCR measurement of transcripts. Separate primers for spliced and unspliced RNA are used with a common downstream primer in excess. PCR products were quantitated using ImageQuant software (Molecular Dynamics).

FIG. 3B is a set of graphs providing a comparison of RT-PCR and microarray data. All values are log₂. Phosphorimager counts for each PCR product were normalized to the average of the two intronless genes to adjust for differences in mRNA levels of the different samples. The normalized values from PCR were treated as intensity measures for intron or splice junction array probes. The ratios for total gene-derived (exon 2-containing) RNA were obtained from the ratios of the sums of the normalized spliced and unspliced counts for each gene. The PM Index derived from the PCR data represents counts in unspliced RNA divided by counts in spliced RNA in the same lane. Numbers next to gene names indicate the distance from bp to 3′ ss in nucleotides.

FIG. 4A shows a strategy for probes covering an alternatively spliced gene. Red lines show splicing events in type 1 cells, green for type 2, yellow for both.

FIG. 4B is an idealized array result. Common features measure total mRNA from the gene.

FIG. 5 is a list of exemplary genes for analysis using an array of the invention.

FIG. 6 is a schematic diagram showing detection of CD44 alternative splicing using oligonucleotide arrays. Boxes represent exons and the numbers represent the log ratio that describes the relative levels of CD44 splice variants in two cell lines.

### DEFINITIONS

The terms “nucleic acid” (e.g., as in “nucleic acid probe”), “nucleic acid molecule” and “polynucleotide” are used interchangeably and refer to a polymeric form of nucleotides of any length, either deoxyribonucleotides or ribonucleotides, or analogs thereof. Thus, these terms include, but are not limited to, single-, double-, or multi-stranded DNA or RNA, genomic DNA, cDNA, DNA-RNA hybrids, nucleic acid having the sequence of a sense strand, antisense nucleic acid, and peptide nucleic acid (PNA), as well as polymers comprising purine and pyrimidine bases or other natural, chemically or biochemically modified, non-natural, or derivatized nucleotide bases. These comprise intronic and exonic sequences Polynucleotides may have any three-dimensional structure, with the proviso that when used as probes the three-dimensional structure is amenable to selective hybridization to a nucleic acid of at least partially complementary sequence. Non-limiting examples of polynucleotides include those having a sequence of a gene, a gene fragment, exons, introns, splice junctions, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidites and thus can be an oligodeoxynucleoside phosphoramidate or a mixed phosphoramidate-phosphodiester oligomer. Peyrottes et al. (1996) Nucl. Acids Res. 24:1841-1848; Chaturvedi et al. (1996) Nucl. Acids Res. 24:2318-2323. A polynucleotide may comprise modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a support (e.g., to a solid or semi-solid support, to a support for use as an array, and the liked). Polynucleotides can be provided in a variety of forms, e.g., associated with an array.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term polynucleotide sequence is the alphabetical representation of a polynucleotide molecule. This alphabetical representation can be input into databases in a computer having a central processing unit and used for bioinformatics applications such as functional genomics and homology searching.

An “intron” is generally a genomic nucleic acid sequence that is removed during mRNA splicing in the generation of a particular spliced mRNA variant. In other words, within one spliced variant of a gene, an intron is removed by mRNA splicing.

An “exon” is generally a genomic nucleic acid sequence that is retained during mRNA splicing in the generation of a particular spliced mRNA variant. In other words, within one spliced variant of a gene, an exon is retained by mRNA splicing.

It is understood that “intron” and “exon” are relative with respect to a particular mRNA spliced variant, and that an exon of one spliced variant may be an intron of another, and vice versa. However, within one spliced variant, an “intron” cannot be an “exon” and vice versa. These terms “intron” and “exon” are used herein for convenience and clarity and are not meant to be limiting.

A “splice junction” is the junction between two exons within a particular spliced variant of a gene. The splice junction is a product of mRNA splicing, and the contiguous sequence bridging the splice junction (e.g., a contiguous sequence extending from the 3′ end of a first exon, across the junction, and to the 5′ end of a second exon) is not present in the corresponding genomic DNA.

A “splice site” is a site between an exon and an adjacent intron in unspliced mRNA, and can either be at the 5′ end an intron, or the 3′ end of an intron.

“Constitutively spliced exon” refers to an exon that is present in all mRNA spliced variants of a selected gene.

A “coding sequence” or a sequence which “encodes” a selected polypeptide, is a nucleic acid molecule which is transcribed (in the case of DNA) and translated (in the case of mRNA) into a polypeptide, for example, in vivo when placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from viral, procaryotic or eucaryotic mRNA, genomic DNA sequences from viral or procaryotic DNA, and even synthetic DNA sequences. A transcription termination sequence may be located 3′ to the coding sequence. Other “control elements” may also be associated with a coding sequence. A DNA sequence encoding a polypeptide can be optimized for expression in a selected cell by using the codons preferred by the selected cell to represent the DNA copy of the desired polypeptide coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. Also encompassed are polypeptide sequences which are immunologically identifiable with a polypeptide encoded by the sequence.

“Operably linked” refers to an arrangement of elements wherein the components so described are configured so as to perform their usual function. Thus, a given promoter that is operably linked to a coding sequence (e.g., a reporter expression cassette) is capable of effecting the expression of the coding sequence when the proper enzymes are present. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

Techniques for determining nucleic acid and amino acid “sequence identity” also are known in the art. Typically, such techniques include determining the nucleotide sequence of the mRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by =HIGH SCORE; Databases non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

Alternatively, homology can be determined by hybridization of polynucleotides under conditions that form stable duplexes between homologous regions, followed by digestion with single-stranded-specific nuclease(s), and size determination of the digested fragments. Two DNA, or two polypeptide sequences are “substantially homologous” to each other when the sequences exhibit at least about 80%-85%, preferably at least about 85%-90%, more preferably at least about 90%-95%, and most preferably at least about 95%-98% sequence identity over a defined length of the molecules, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

Two nucleic acid molecules are considered to “selectively hybridize” or “specifically hybridize”, which terms are used interchangeably, as described herein when the molecule hybridize to one another preferentially over nucleic acid molecules having a different nucleotide sequence. The degree of sequence identity between two nucleic acid molecules affects the efficiency and strength of hybridization events between such molecules. A partially identical nucleic acid sequence will at least partially inhibit a completely identical sequence from hybridizing to a target molecule. Inhibition of hybridization of the completely identical sequence can be assessed using hybridization assays that are well known in the art (e.g., Southern blot, Northern blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a target nucleic acid sequence, and then by selection of appropriate conditions the probe and the target sequence “selectively hybridize,” or bind, to each other to form a hybrid molecule. A nucleic acid molecule that is capable of hybridizing selectively to a target sequence under “moderately stringent” typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/target hybridization where the probe and target have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., formamide, dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.)

A first polynucleotide is “derived from” a second polynucleotide if it has the same or substantially the same basepair sequence as a region of the second polynucleotide, its cDNA, complements thereof, or if it displays sequence identity as described above.

For convenience and clarity, reference to a probe specific for an mRNA is also meant to refer to a probe specific for a cDNA derived from that mRNA species.

In the present invention, when a sequence “derived from a gene” the sequence need not be explicitly from the sequence as it exists in nature, but instead includes synthetic and natural sequences that use the sequence of a naturally-occurring gene sequence as a template. For example, a probe for a splice junction, intron, or exon sequence may be specific for the sequence of an mRNA having the splice junction, intron, or exon, or may be specific for a cDNA generated from such mRNA.

“Substantially purified” general refers to isolation of a substance (compound, polynucleotide, protein, polypeptide, polypeptide composition) such that the substance comprises the majority percent of the sample in which it resides. Typically in a sample a substantially purified component comprises 5.0%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

“Target sequence” and “target nucleic acid” is meant to refer to nucleic acid in a sample having a sequence to which a probe will selectively hybridize.

Before the present subject invention is described further, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a probe” includes a plurality of such probes and reference to “the target sequence” includes reference to one or more target sequences and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The invention features an array comprising sets of nucleic acid probes for detection of gene products that are produced by mRNA splicing of a selected gene, wherein each probe set is specific for a selected gene, and wherein each probe set comprises a splice junction probe and either an intron probe or an exon probe. The splice junction probe hybridizes selectively to a sequence corresponding to a pre-selected, non-genomic sequence present in a product of mRNA splicing, whereas the intron probe hybridizes selectively to a sequence corresponding to an intronic sequence present in unspliced mRNA and the exon probe hybridizes selectively to a sequence corresponding to an exonic sequence of the selected gene. Either the intron or exon probe can serve as an internal control. The invention also features methods of using the array to analyze mRNA splice products in a sample.

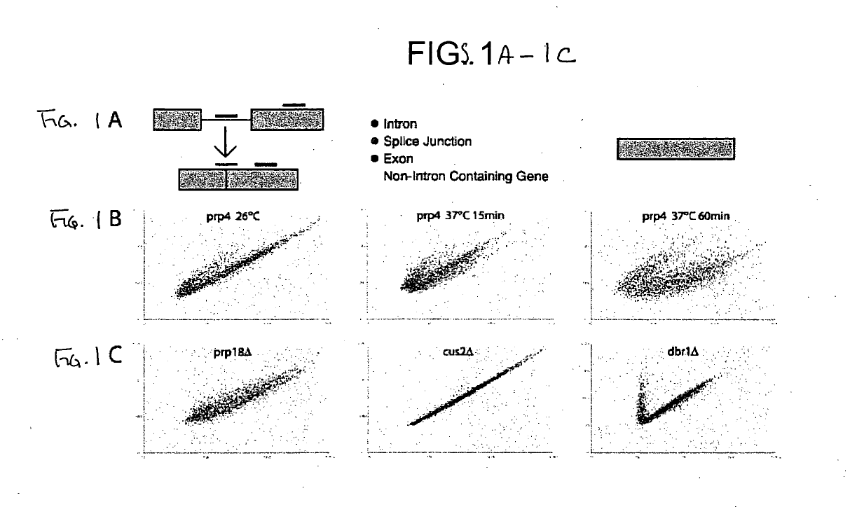

An exemplary embodiment of invention can be best understood with reference to the schematic of FIG. 1A, which shows an exemplary design of an array of the invention. The array comprises sets of probes specific for mRNA splice products of a selected gene. Each set of probes on the array contains at least two probes—a splice junction (SJ) probe and either an intron probe or an exon probe. The intron probe detects the presence of a sequence corresponding to an unspliced RNA and intron lariats formed by splicing reactions. The SJ probe selectively detects properly spliced mRNA products. The exon probe detects a sequence present in both spliced and unspliced RNAs, and may serves as an internal control. In one embodiment, when the exon probe detects a constitutively spliced exon, SJ probe hybridization data may be internally normalized (i.e. within a selected gene) using exon probe hybridization data for the selected gene. In another embodiment, SJ probe hybridization data may be internally normalized using intron probe hybridization data for the selected gene. In a further embodiment, data is normalized using one or more probes that are specific for one or more intronless genes (non-intron containing genes).

Thus, for each gene for which splicing is to be monitored, the invention provides a set of at least two, and in some embodiments three, specific probes: 1) an SJ probe and 2) either an intron probe or an exon probe. The SJ probe is present in all embodiments of the splicing assay of the invention, and discriminates between spliced and unspliced RNA. The intron probe also discriminates between spliced and unspliced RNA, and both the intron probe and the exon probe may function as an internal control, depending on the design of the experiment.

In one embodiment, each of the probes is present in an array at a defined location. Hybridization of each of the probes to nucleic acid in a sample can be detected in a variety of ways, including through detection of a detectable signal (such as a fluorescent probe) associated with nucleic acid of a sample to be analyzed.

The relationship of the detectable signals associated with hybridization to each of the SJ and intron or exon probes can be examined to determine how various biological conditions influence RNA splicing of the individual genes in the context of all the genes in the genome. The technique is readily extended to any organism for which the sequence of the genomic DNA, and the sequences comprising the splice junctions within the various forms of the mature mRNA are known.

The invention can be applied to many uses, including for research purposes to study the effects that mutations, disease, or environmental conditions may have on gene expression that is controlled at the level of RNA processing. The invention can also be used to investigate the nature of various splicing defects as well as expression of various RNA isoforms present in different tissue types of higher organisms. Because the invention employs both genomic and non-genomic sequences to monitor RNA splicing, it can detect multiple isoforms and their relative proportions in a single sample. This feature could increase the invention's value as a diagnostic tool, if for example, the presence of a particular RNA isoform in any amount, was responsible for a particular disease state.

Each aspect of the invention will now be described in more detail.

Array Structure

The arrays of the subject invention have a plurality of probe oligonucleotide spots stably associated with a surface of a solid support. Each oligonucleotide spot on the array comprises an oligonucleotide probe composition of known identity, usually of known sequence, as described in greater detail below. The oligonucleotide spots on the array may be any convenient shape, but will typically be circular, elliptoid, oval or some other analogously curved shape. The density of the spots on the solid surface is at least about 5/mm² and usually at least about 10/mm² to 30/mm² more usually about 28/mm² (or about 2800/cm²) but does not exceed about 1000/mm², and usually does not exceed about 500/mm² or 400/mm², and more usually does not exceed about 300/mm². The spots may be arranged in a spatially defined and physically addressable manner, in any convenient pattern across or over the surface of the array, such as in rows and columns so as to form a grid, in a circular pattern, and the like, where generally the pattern of spots will be present in the form of a grid across the surface of the solid support.

In the subject arrays, the spots of the pattern are stably associated with the surface of a solid support, where the support may be a flexible or rigid support. By “stably associated” it is meant that the oligonucleotides of the spots maintain their position relative to the solid support under hybridization and washing conditions. As such, the oligonucleotide members which make up the spots can be non-covalently or covalently stably associated with the support surface based on technologies well known to those of skill in the art. Examples of non-covalent association include non-specific adsorption, binding based on electrostatic (e.g. ion-ion pair interactions), hydrophobic interactions, hydrogen bonding interactions, specific binding through a specific binding pair member covalently attached to the support surface, and the like. Examples of covalent binding include covalent bonds formed between the spot oligonucleotides and a functional group present on the surface of the rigid support, e.g. —OH, where the functional group may be naturally occurring or present as a member of an introduced linking group, as described in greater detail below. Alternatively, the oligonucleotides can be stably associated by virtue of a physical characteristic of the assay support, e.g., by providing for a well or other barrier that restricts movement of oligonucleotides from one spot to another, and prevents significant loss of oligonucleotides from the assay substrate.

As mentioned above, the array is present on either a flexible or rigid substrate. By flexible is meant that the support is capable of being bent, folded or similarly manipulated without breakage. Examples of solid materials which are flexible solid supports with respect to the present invention include membranes, flexible plastic films, and the like. By rigid is meant that the support is solid and does not readily bend, i.e. the support is not flexible. As such, the rigid substrates of the subject arrays are sufficient to provide physical support and structure to the polymeric targets present thereon under the assay conditions in which the array is employed, particularly under high throughput handling conditions. Furthermore, when the rigid supports of the subject invention are bent, they are prone to breakage.

The solid supports upon which the subject patterns of spots are presented in the subject arrays may take a variety of configurations ranging from simple to complex, depending on the intended use of the array. Thus, the substrate could have an overall slide or plate configuration, such as a rectangular or disc configuration. In many embodiments, the substrate will have a rectangular cross-sectional shape, having a length of from about 10 mm to 200 mm, usually from about 40 to 150 mm and more usually from about 75 to 125 mm and a width of from about 10 mm to 200 mm, usually from about 20 mm to 120 mm and more usually from about 25 to 80 mm, and a thickness of from about 0.01 mm to 5.0 mm, usually from about 0.1 mm to 2 mm and more usually from about 0.2 to 1 mm. Thus, in one embodiment the support may have a micro-titre plate format, having dimensions of approximately 125×85 mm.

The substrates of the subject arrays may be fabricated from a variety of materials. The materials from which the substrate is fabricated should ideally exhibit a low level of non-specific binding during hybridization events. In many situations, it will also be preferable to employ a material that is transparent to visible and/or UV light. For flexible substrates, materials of interest include: nylon, both modified and unmodified, nitrocellulose, polypropylene, and the like, where a nylon membrane, as well as derivatives thereof, is of particular interest in this embodiment. For rigid substrates, specific materials of interest include: glass; plastics, e.g. polytetrafluoroethylene, polypropylene, polystyrene, polycarbonate, and blends thereof, and the like; metals, e.g. gold, platinum, and the like; etc.

The substrates of the subject arrays comprise at least one surface on which the pattern of spots is present, where the surface may be smooth or substantially planar, or have irregularities, such as depressions or elevations. The surface on which the pattern of spots is present may be modified with one or more different layers of compounds that serve to modify the properties of the surface in a desirable manner. Such modification layers, when present, will generally range in thickness from a monomolecular thickness to about 1 mm, usually from a monomolecular thickness to about 0.1 mm and more usually from a monomolecular thickness to about 0.001 mm. Modification layers of interest include: inorganic and organic layers such as metals, metal oxides, polymers, small organic molecules and the like. Polymeric layers of interest include layers of: peptides, proteins, polynucleic acids or mimetics thereof, e.g. peptide nucleic acids and the like; polysaccharides, phospholipids, polyurethanes, polyesters, polycarbonates, polyureas, polyamides, polyethyleneamines, polyarylene sulfides, polysiloxanes, polyimides, polyacetates, polyacrylamides, and the like, where the polymers may be hetero- or homopolymeric, and may or may not have separate functional moieties attached thereto, e.g. conjugated.

The total number of spots on the substrate will vary depending on the number of different oligonucleotide spots (oligonucleotide probe compositions) one wishes to display on the surface, as well as the number of control spots, orientation spots, calibrating spots and the like, as may be desired depending on the particular application in which the subject arrays are to be employed. Generally, the pattern present on the surface of the array will comprise at least about 10 distinct oligonucleotide spots, usually at least about 20 distinct oligonucleotide spots, and more usually at least about 50 distinct oligonucleotide spots, where the number of oligonucleotide spots may be as high as 20,000 or higher, but will usually not exceed about 15,000 distinct oligonucleotide spots, and more usually will not exceed about 9,000 distinct oligonucleotide spots and in many instances will not exceed about 1,000. In many embodiments, it is preferable to have each distinct oligonucleotide spot or probe composition presented in duplicate, i.e. so that there are two spots for each distinct oligonucleotide probe composition of the array. In certain embodiments, the number of spots will range from about 200 to 600.

In the arrays of the subject invention (particularly those designed for use in high throughput applications, such as high throughput analysis applications), a single pattern of oligonucleotide spots may be present on the array or the array may comprise a plurality of different oligonucleotide spot patterns, each pattern being as defined above. When a plurality of different oligonucleotide spot patterns are present, the patterns may be identical to each other, such that the array comprises two or more identical oligonucleotide spot patterns on its surface, or the oligonucleotide spot patterns may be different, e.g. in arrays that have two or more different types of target nucleic acids represented on their surface, e.g an array that has a pattern of spots corresponding to human genes and a pattern of spots corresponding to mouse genes. Where a plurality of spot patterns are present on the array, the number of different spot patterns is at least 2, usually at least 6, more usually at least 24 or 96, where the number of different patterns will generally not exceed about 384.

Where the array comprises a plurality of oligonucleotide spot patterns on its surface, preferably the array comprises a plurality of reaction chambers, wherein each chamber has a bottom surface having associated therewith an pattern of oligonucleotide spots and at least one wall, usually a plurality of walls surrounding the bottom surface. Of particular interest in many embodiments are arrays in which the same pattern of spots in reproduced in 24 or 96 different reaction chambers across the surface of the array.

Within any given pattern of spots on the array, there may be a single spot that corresponds to a given target or a number of different spots that correspond to the same target, where when a plurality of different spots are present that correspond to the same target, the probe compositions of each spot that corresponds to the same target may be identical of different. In other words, a plurality of different targets are represented in the pattern of spots, where each target may correspond to a single spot or a plurality of spots, where the oligonucleotide probe composition among the plurality of spots corresponding to the same target may be the same or different. Where a plurality of spots (of the same or different composition) corresponding to the same target is present on the array, the number of spots in this plurality will be at least about 2 and may be as high as 10, but will usually not exceed about 5. The number of different targets represented on the array is at least about 2, usually at least about 10 and more usually at least about 20, where in many embodiments the number of different targets, e.g. genes, represented on the array is at least about 50. The number of different targets represented on the array may be as high as 5000 or higher, but will usually not exceed about 1000 and more usually will not exceed about 700. A target is considered to be represented on an array if it is able to hybridize to one or more probe compositions on the array. For each gene, at least 1, usually at least 2, more usually at least 5, even more usually at least 10, and up to 50 or 100 or more splice junctions probes can be represented on an array.

The total amount or mass of oligonucleotides present in each spot will be sufficient to provide for adequate hybridization and detection of target nucleic acid during the assay in which the array is employed. Generally, the total mass of oligonucleotides in each spot will be at least about 0.1 ng, usually at least about 0.5 ng and more usually at least about 1 ng, where the total mass may be as high as 1000 ng or higher, but will usually not exceed about 20 ng and more usually will not exceed about 10 ng. The copy number of all of the oligonucleotides in a spot will be sufficient to provide enough hybridization sites for target molecule to yield a detectable signal, and will generally range from about 0.01 fmol to 50 fmol, usually from about 0.05 fmol to 20 fmol and more usually from about 0.1 fmol to 5 fmol. The molar ratio or copy number ratio of different oligonucleotides within each spot may be about equal or may be different, wherein when the ratio of unique oligonucleotides within each spot differs, the magnitude of the difference will usually be at least 2 to 10 fold but will generally not exceed about 100 fold. Where the spot has an overall circular dimension, the diameter of the spot will generally range from about 10 to 5,000 μm, usually from about 20 to 1,000 μm and more usually from about 50 to 500 μm. The surface area of each spot is at least about 100 μm², usually at least about 400 μm² and more usually at least about 800 μm², and may be as great as 25 mm² or greater, but will generally not exceed about 5 mm², and usually will not exceed about 1 mm².

In one embodiment of the invention, each of the oligonucleotide spots in the array comprising the oligonucleotide probe compositions correspond to one or more splice variants of one or more selected genes, which selected genes may be of a particular class or type. For example, the probes can provide for detection of splice variants of genes that share some common characteristic or can be grouped together based on some common feature, such as species of origin, tissue or cell of origin, functional role, disease association, and the like. For example, each of different target nucleic acids that correspond to the different probe spots on the array can be of the same type, e.g., comprise coding sequences for the same or same type of gene. For example, the arrays can comprise probes for target sequence of human genes, genes implicated in cancer (e.g., oncogenes, tumor suppressor genes, and the like), genes implicated in apoptosis, genes involved in neurogenesis, stress genes, signal transduction genes, and the like.

With respect to the oligonucleotide probes that correspond to a particular type or kind of gene, type or kind can refer to a plurality of different characterizing features, where such features include: species specific genes, where specific species of interest include eukaryotic species, such as mice, rats, rabbits, ungulates (e.g., pigs, cows, goats), primates (e.g., monkeys, chimpanzees, humans), and the like; function specific genes, where such genes include oncogenes, apoptosis genes, cytokines, receptors, protein kinases, etc.; genes specific for or involved in a particular biological process, such as apoptosis, differentiation, stress response, aging, proliferation, etc.; cellular mechanism genes, e.g. cell-cycle, signal transduction, metabolism of toxic compounds, etc.; disease associated genes, e.g. genes involved in cancer, schizophrenia, diabetes, high blood pressure, atherosclerosis, viral-host interaction and infection diseases, etc.; location specific genes, where locations include organ, such as heart, liver, prostate, lung etc., tissue, such as nerve, muscle, connective, etc., cellular, such as axonal, lymphocytic, etc, or subcellular locations, e.g. nucleus, endoplasmic reticulum, Golgi complex, endosome, lysosome, peroxisome, mitochondria, cytoplasm, cytoskeleton, plasma membrane, extracellular space, chromosome-specific genes; specific genes that change expression level over time, e.g. genes that are expressed at different levels during the progression of a disease condition, such as prostate genes which are induced or repressed during the progression of prostate cancer.

In addition to the oligonucleotide spots comprising the oligonucleotide probe compositions (i.e. oligonucleotide probe spots), the subject arrays may comprise one or more additional spots of polynucleotides which do not correspond to target nucleic acids as defined above, such as target nucleic acids of the type or kind of gene represented on the array in those embodiments in which the array is of a specific type. In other words, the array may comprise one or more spots that are made of non-“unique” oligonucleotides or polynucleotides, e.g., common oligonucleotides or polynucleotides. For example, spots comprising genomic DNA may be provided in the array, where such spots may serve as orientation marks. Spots comprising plasmid and bacteriophage genes, genes from the same or another species which are not expressed and do not cross hybridize with the cDNA target, and the like, may be present and serve as negative controls. In addition, spots comprising a plurality of oligonucleotides complimentary to housekeeping genes and other control genes from the same or another species may be present, which spots serve in the normalization of mRNA abundance and standardization of hybridization signal intensity in the sample assayed with the array. Orientation spots may also be included on the array, where such spots serve to simplify image analysis of hybrid patterns. These latter types of spots are distinguished from the oligonucleotide probe spots, i.e. they are non-probe spots.

The array may further comprise mismatch control probes. Mismatch controls may be provided for the probes to the target genes, for expression level controls or for normalization controls. Mismatch controls are oligonucleotide probes identical to their corresponding test or control probes except for the presence of one or more mismatched bases. A mismatched base is a base selected so that it is not complementary to the corresponding base in the target sequence to which the probe would otherwise specifically or selectively hybridize. One or more mismatches are selected such that under appropriate hybridization conditions (e.g. stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a 20 mer, a corresponding mismatch probe will have the identical sequence except for a single base mismatch (e.g., substituting a G, a C or a T for an A) at any of positions 6 through 14 (the central mismatch).

Mismatch probes thus provide a control for non-specific binding or cross-hybridization to a nucleic acid in the sample other than the target to which the probe is directed. Mismatch probes thus indicate whether a hybridization is specific or not. For example, if the target is present the perfect match probes should be consistently brighter than the mismatch probes. In addition, if all central mismatches are present, the mismatch probes can be used to detect a mutation. Finally, the difference in intensity between the perfect match and the mismatch probe (I(PM)-I(MM)) provides a good measure of the concentration of the hybridized material.

The subject arrays can be prepared using any convenient means. One means of preparing the subject arrays is to first synthesize the oligonucleotides for each spot and then deposit the oligonucleotides as a spot on the support surface. The oligonucleotides may be prepared using any convenient methodology, such as automated solid phase synthesis protocols, and like, where such techniques are well known to those of skill in the art.

The prepared oligonucleotides may be spotted on the support using any convenient methodology, including manual techniques, e.g. by micro pipette, ink jet, pins, etc., and automated protocols, where the different oligonucleotides of each spot can be mixed together as described above and spotted or spotted separately in the same spot location in a sequential fashion. Of particular interest is the use of an automated spotting device, such as the Beckman Biomek 2000 (Beckman Instruments) or the Omnigrid (Genemachines Inc.).

Some embodiments of the invention may involve arrays of beads in which each oligonucleotide is attached to a bead of different physical composition which does not interfere with the hybridization of the probe. In this case the identity of the bead (and thus the identity of the oligonucleotide) is not given by its position in a two-dimensional array but by its association with a bead of a specified physical type (e.g., a bead having a specific detectable label, or a specified size, and the like).

Oligonucleotide Probes of the Arrays

For each selected gene, at least two probes corresponding to sequences of the gene are spotted onto the array. The two probes are an SJ probe and either an intron or an exon probe. All three types of probe may be spotted. Either the exon probe or the intron probe can serve as an internal control for the SJ probe. An exon probe can serve as an internal control probe for the SJ probe if the exon is constitutively spliced. An intron probe can function as an internal control for a particular SJ probe if the intron probe selectively hybridizes to an intron removed in the production of the particular splice junction to which the SJ probe selectively hybridizes (e.g. such that removal of the intron results in an increase in the hybridization signal of the SJ probe and a decrease in the signal of the intron probe. For each selected gene preferably at least 5 probes, more preferably at least 10 probes, even more preferably at least 20, and most preferably at least about 50 or at least about 100 different probes are spotted on the array. In general, probes are represented as a spot of an oligonucleotide on the surface of a support. Several types of probe are provided by the invention, including a splice junction probe, an intron probe, an exon probe and a control probe. Probe sequences allow for hybridization to experimental nucleic acid samples, particularly at high stringency. A description of the general probe composition is presented below, followed by a description of each of the probe types.

Each oligonucleotide spot on the surface of a substrate is made up of an oligonucleotide probe. By “oligonucleotide probe” is meant an oligonucleotide capable of hybridizing to a selected distinct or different region of the selected gene to which it corresponds, i.e. the selected gene corresponding to the spot in which the oligonucleotide is positioned. For each selected gene, several oligonucleotide spots may be deposited including at least one SJ probe, and at least one of an intron probe or at least one exon probe.

By “capable of hybridizing to distinct or different regions” is meant that the different oligonucleotides of the probe hybridize to a different stretch of nucleotide residues in the selected gene, where the different stretches or regions of the nucleic acid sample may be continuous, separated by one or more nucleotide residues, or overlapping but physically belong to the same target molecule. The different regions of the target nucleic acid of particular interest are splice junctions, introns, and exons.

With respect to probes that do not correspond to the same target, the oligonucleotides are chosen so that each distinct oligonucleotide is not homologous with any other distinct oligonucleotide. In other words, each distinct oligonucleotide of a probe does not cross-hybridize with, or have the same sequence as, any other distinct oligonucleotide on of any probe corresponding to a different target.

As such, the sense or anti-sense nucleotide sequence of each oligonucleotide of a probe will have less than 90% homology, usually less than 85% homology, and more usually less than 80% homology with any other different oligonucleotide of a probe corresponding to a different target of the array, where homology is determined by sequence analysis comparison using the FASTA program using default settings. In general, the sequence of oligonucleotides in the probe are not conserved sequences found in a number of different genes (at least two), where a conserved sequence is defined as a stretch of from about 15 to 150 nucleotides which have at least about 90% sequence identity, where sequence identity is measured as above. The length of the oligonucleotide will be shorter than the mRNA to which it corresponds. However, where more than one probe of the array corresponds to the same selected gene (e.g., to provide for multiple data points for a selected gene), the same oligonucleotide may be present in two or more of these probes that all correspond to the same target gene. In other words, among such probe that correspond to the same target gene, such probe may have one or more oligonucleotides in common (e.g., shared exon or intron probes).

The oligonucleotides of the subject probe will generally have a length of from about 15 to 150 nt, usually from 25 to 100 nt, and more usually 30 to 70 nt.

All oligonucleotides corresponding to a selected gene, and preferably all oligonucleotides on an array should have substantially the same melting temperature to the target nucleic acid. In other words, the melting temperature or Tₘ of any double stranded complex formed between any one oligonucleotide and the target should not be substantially different from the Tₘ of any other double stranded complex formed between the target and any other oligonucleotide. By “substantially the same” is meant that any difference in Tₘ will not exceed more than 30 degrees C., usually not more than about 20 degrees C. and more usually not more than about 10 degrees C.

In general, the oligonucleotides of each probe are further characterized by having a GC content of from about 35% to 80%. The oligonucleotides are also characterized by the substantial absence of secondary structures and long homopolymeric stretches, e.g. polyA stretches, such that in any give homopolymeric stretch, the number of contiguous identical nucleotide bases does not exceed 5. In special cases where a specific RNA sequence such as a splice junction is targeted, the general characteristics of the probe will match those of the target including G+C content and the presence of homopolymeric stretches.

Depending on the nature of the nucleic acid sample, the oligonucleotide of a probe may bind to the same nucleic acid strand or to different nucleic strands. Thus, where the target is single stranded, i.e. mRNA or cDNA, the oligonucleotides will bind to the same target strand. In contrast, where the target is double stranded, such as double stranded cDNA, the oligonucleotides may bind to the same strand or to different strands, e.g. one oligonucleotide may bind to the sense strand and one may bind to the anti-sense strand.

The oligonucleotide probe that makes up each oligonucleotide spot on the array will be substantially, usually completely, free of non-nucleic acids, i.e. the probe will not comprise non-nucleic acid biomolecules found in cells, such as proteins, lipids, and polysaccharides. In other words, the oligonucleotide spots of the arrays are substantially, if not entirely, free of non-nucleic acid cellular constituents.

The oligonucleotide probes may be nucleic acid, e.g. RNA, DNA, or nucleic acid mimetics, e.g. such as nucleic acids comprising non-naturally occurring heterocyclic nitrogeneous bases, peptide-nucleic acids, locked nucleic acids (see Singh & Wengel, Chem. Commun. (1998) 1247-1248); and the like.

Splice-Junction (SJ) Probes

An SJ probe is a polynucleotide that specifically and selectively hybridizes to a region spanning a splice junction, i.e., a site of splicing of a first exon to a second exon. A splice junction probe sequence is thus not present in an unspliced gene product. Stated differently, the SJ probe will not hybridize to significant or detectable levels to an unspliced mRNA gene product or to cDNA produced from such an unspliced mRNA gene product. As such, an SJ probe spans an exon/exon junction and is designed to have a length and/or GC-richness that minimizes hybridization to two exon sequences that have not been spliced together.

Typically, SJ probes are approximately 15 to 60 nucleotides in length, preferably 35-45 and most preferably about 40 nucleotides in length. SJ probes typically span the splice junction such that approximately one half of the length of the oligonucleotide will hybridize to sequences adjacent to the splice junction in one exon and the other half of the length of the oligonucleotide will hybridize to sequences adjacent to the splice junction in the other exon. SJ probe characteristics may be adjusted altering a number of physical features of the SJ probe, for example by altering GC-content, length of the probe, portion of the probe directed against each exon, and by substituting nucleotides with modified bases, e.g. inosine. An effective SJ probe may also be designed empirically, where probes of several different lengths and compositions may be tested for efficacy, and effective SJ probes may be tiled across a splice junction. Hybridization conditions may also be altered to adjust the effectiveness of a particular SJ probe.

In the compositions and methods of this invention, SJ probes are pre-selected, i.e., the SJ probe is designed for a particular, pre-selected target sequence. SJ probes can pre-selected using many methods which may be used alone or in combination. Exemplary methods for identification and selection of SJ probe sequences, which methods are not intended to be limiting, may use bioinformatics and/or experimental approaches. One such bioinformatics approach involves the prediction of genes from raw nucleic acid sequence. For example, several gene prediction programs such as Genscan, mzef, fgenesh etc. may be used to predict intron-exon boundaries for genes, and thus may be used to pre-select SJ probes. These programs have usually been trained with existing sets of experimentally defined splice sites, and many have recently been reviewed (Burset, et al., Genomics 34: 353-367 (1996) and Guigo et al Genome Res. (2000) 10:1631-42). Another bioinformatics approach for identifying splice junctions involves comparing raw genomic sequences of one species to that of another species. Exon sequences are normally evolutionarily less divergent than intron sequences and this information can be used to pinpoint coding sequences.

Further methods for identifying SJ sequence rely on experimental evidence. Such methods may rely on bioinformatics tools, but it is understood that the methods may be performed manually. One particularly successful method for identifying SJ sequences relies on using experimental sequences derived from cDNAs (i.e. from spliced mRNA). cDNA sequences in the form of expressed sequence tags (EST), partial cDNA sequences, sequence of reverse transcriptase PCR products or full length cDNA sequences may be cross-compared and/or compared to raw genomic sequences using sequence alignment programs such as BLAST. Once sequences are aligned, annotation and pre-selection of splice junctions is done manually or programmatically. Such methods are well known in the art (e.g. Kan et al (2001) Genome Research 11:889-900). One of skill in the art may recognize that there are several other ways to predict splice junctions.

Exon Probes

An exon probe is a polynucleotide that specifically and selectively hybridizes to a target sequence of an exon of a particular spliced variant. An exon probe may also hybridize to a portion of an intron sequence, with the proviso that the exon probe can hybridize to substantially the same level to a spliced or unspliced mRNA gene product or to cDNA produced from such gene products. An exon probe for one spliced variant of a selected gene may be an intron probe for a different spliced variant of the selected gene.

In some embodiments, the exon probe can serve as an internal control probe, particularly where the exon is constitutively spliced, i.e., is present in all detectable mRNA spliced products, or is known to be present in the mRNA spliced product(s) of interest for analysis. When an exon probe is an internal control probe, it can serve as an internal standard for SJ probe hybridization. Exon probes can be predicted using the software and experimental methodologies described above.

In other embodiments, the exon probe can contribute to the estimation of the representation of one particular spliced variant. If the probe targets an exon that is variably or alternatively included in a specific isoform, that exon probe may uniquely represent that spliced variant and be combined with the data from splice junction probes that uniquely identify the same splice variant.

Intron Probes

An intron probe is a polynucleotide that specifically and selectively hybridizes to a target sequence positioned between exons of an mRNA gene product. Intron probes may also hybridizes to a portion of an exon sequence, with the proviso that the intron probe does not hybridize to a particular properly spliced mRNA gene product, i.e., splicing removes sufficient intron probe target sequence such that the intron probe will not hybridize to significant or detectable levels to the spliced mRNA gene product (or to cDNA produced from such a gene product). As recited above, an intron probe for one spliced variant of a selected gene may be an exon probe for another spliced variant of the selected gene.

In some embodiments, the intron probe can serve as an internal control probe. When an intron probe hybridizes with an intron that is spliced out to form a particular splice junction, it may be used as an internal control probe i.e. it can serve as an internal standard for SJ probe hybridization in that the amount of SJ probe hybridization should vary inversely to the amount of intron probe hybridization. An intron probe may be predicted using the software and experimental methodologies described above.

Control Probe

An internal control probe is a sequence from a selected gene for which the efficiency or rate of splicing is known. Particularly suitable internal control probes represent a constitutively spliced sequence, e.g., an exonic sequence that is present in all mRNA spliced variants from a gene, a splice junction known to be present in all mRNA splice variants from a gene, or other sequence that is at a known amount in mRNA spliced products.

Intronless gene probes can also serve as a control to indicate that the hybridization assay is functioning properly, and/or to provide for normalization of assays. Intronless gene probes are selected so as to specifically and selectively hybridize to a target sequence of an intronless gene, with little or no detectable hybridization to a target sequence of any of the SJ, intron, or exon probes. Constitutively spliced exon or splice junction probes may also serve as a normalization probe. Constitutively spliced and constitutively expressed genes may serve as hybridization controls.

Generation of Labeled Target Nucleic Acid, Hybridization and Detection

The subject arrays find use in a variety of different applications in which one is interested in detecting alternative splicing or rate of splicing. In general, the device will be contacted with the sample suspected of containing the splice variants under conditions sufficient for binding of any splice variants present in the sample to complementary oligonucleotide probes present on the array. Generally, the sample will be a fluid sample and contact will be achieved by introduction of an appropriate volume of the fluid sample onto the array surface, where introduction can be through delivery ports, direct contact, deposition, and the like.

Targets may be generated by methods known in the art. mRNA can be labeled and used directly as a target, or converted to a labeled cDNA target. Usually, mRNA is labeled directly using chemically, photochemically or enzymatically activated labeling compounds, such as photobiotin (Clontech, Palo Alto, Calif.), Dig-Chem-Link (Boehringer), and the like. Generally, methods for generating labeled cDNA probes include the use of oligonucleotide primers. Primers that may be employed include oligo dT, random primers, e.g. random hexamers and gene specific primers. Where gene specific primers are employed, the gene specific primers are preferably those primers that correspond to the different oligonucleotide spots on the array. Thus, one will preferably employ gene specific primers for each different oligonucleotide that is present on the array, so that if the gene is expressed in the particular cell or tissue being analyzed, labeled target will be generated from the sample for that gene. In this manner, if a particular gene present on the array is expressed in a particular sample, the appropriate target will be generated and subsequently identified.

For each target represented on the array, a single gene specific primer may be employed or a plurality of different gene specific primers may be employed, where when a plurality are used to produce the target. Generally, in preparing the target from template nucleic acid, e.g. mRNA, the gene specific primers will hybridize to a region of the template that is downstream from the region to which the probes are homologous, e.g. to which the probes are complementary or have the same sequence, since the primer sequence will represent a sequence common to all mRNA transcripts. However, in certain embodiments the gene specific primers may be complementary to control oligonucleotide probes (e.g., control exon probes). The cDNA probe can be further amplified by PCR, or can be converted (linearly amplified) using phage coded RNA polymerase transcriptionn of dsDNA (double-stranded DNA).

A variety of different protocols may be used to generate the labeled target nucleic acids, as is known in the art, where such methods typically rely in the enzymatic generation of the labeled target using the initial primer. Labeled primers can be employed to generate the labeled target. Alternatively, label can be incorporated during first strand synthesis or subsequent synthesis, labeling or amplification steps in order to produce labeled target.

It is important to ensure that the array design reflects the strand of the target sequence that is labeled. For example, direct detection of mRNA (the plus strand), requires probes that are complementary to the plus strand (the minus strand). Thus for direct labeling or labeling of any derived plus-strand sequence, the probe array will consist of minus-strand sequences. Where the minus strand is labeled, as in reverse transcription of RNA into cDNA, the probe array will contain the sequence like the original RNA target, or the plus strand.

As mentioned above, following preparation of the target nucleic acid (e.g., from a tissue or cell of interest), the target nucleic acid is then contacted with the array under hybridization conditions, where such conditions can be adjusted, as desired, to provide for an optimum level of specificity in view of the particular assay being performed. Suitable hybridization conditions are well known to those of skill in the art, with exemplary conditions described in “Molecular Cloning: A Laboratory Manual,” second edition, edited by Sambrook, Fritsch, & Maniatis, Cold Spring Harbor Laboratory, (1989). In analyzing the differences in the population of labeled target nucleic acids generated from two or more physiological sources using the arrays described above, each population of labeled target nucleic acids are separately contacted to identical probe arrays or together to the same array under conditions of hybridization, preferably under stringent hybridization conditions, such that labeled target nucleic acids hybridize to complementary probes on the substrate surface.

Where all of the nucleic acid samples having target sequences comprise the same label, different arrays can be used. Alternatively, where the labels used to produce detectably labeled target nucleic acid are different and distinguishable for each of the different samples being assayed, the opportunity arises to use the same array at the same time for each of the different target sequence samples. Examples of distinguishable detectable labels are well known in the art and include: two or more different emission wavelength fluorescent dyes, like Cy3 and Cy5, two or more isotopes with different energy of emission, like ³²P and ³³P, gold or silver particles with different scattering spectra, labels which generate signals under different treatment conditions, like temperature, pH, treatment by additional chemical agents, etc., or generate signals at different time points after treatment. Using one or more enzymes for signal generation allows for the use of an even greater variety of distinguishable labels, based on different substrate specificity of enzymes (alkaline phosphatase/peroxidase).

Following hybridization, non-hybridized labeled nucleic acid is removed from the support surface, conveniently by washing, generating a pattern of hybridized nucleic acid on the substrate surface. A variety of wash solutions are known to those of skill in the art and may be used.

The resultant hybridization patterns of labeled nucleic acids may be visualized or detected in a variety of ways, with the particular manner of detection being chosen based on the particular label of the target nucleic acid, where representative detection means include scintillation counting, autoradiography, fluorescence measurement, calorimetric measurement, light emission measurement, light scattering, and the like.

Following detection or visualization, the hybridization patterns may be compared to identify differences between the patterns. Where arrays in which each of the different probes corresponds to a known gene are employed, any discrepancies can be related to a differential expression of a particular gene in the physiological sources being compared.

The provision of appropriate controls on the arrays permits a more detailed analysis that controls for variations in hybridization conditions, cell health, non-specific binding and the like. Thus, for example, in a preferred embodiment, the hybridization array is provided with normalization controls as described supra. These normalization controls are probes complementary to control sequences added in a known concentration to the sample. Where the overall hybridization conditions are poor, the normalization controls will show a smaller signal reflecting reduced hybridization. Conversely, where hybridization conditions are good, the normalization controls will provide a higher signal reflecting the improved hybridization. Normalization of the signal derived from other probes in the array to the normalization controls thus provides a control for variations in hybridization conditions. Typically, normalization is accomplished by dividing the measured signal from the other probes in the array by the average signal produced by the normalization controls. Normalization may also include correction for variations due to sample preparation and amplification. Such normalization may be accomplished by dividing the measured signal by the average signal from the sample preparation/amplification control probes. The resulting values may be multiplied by a constant value to scale the results.

As indicated above, the subject arrays can include mismatch controls. In a preferred embodiment, there is a mismatch control having a central mismatch for every probe (except the normalization controls) in the array. It is expected that after washing in stringent conditions, where a perfect match would, be expected to hybridize to the probe, but not to the mismatch, the signal from the mismatch controls should only reflect non-specific binding or the presence in the sample of a nucleic acid that hybridizes with the mismatch: Where both the probe in question and its corresponding mismatch control both show high signals, or the mismatch shows a higher signal than its corresponding test probe, there is a problem with the hybridization and the signal from those probes is ignored. The difference in hybridization signal intensity between the target specific probe and its corresponding mismatch control is a measure of the discrimination of the target-specific probe. Thus, in a preferred embodiment, the signal of the mismatch probe is subtracted from the signal from its corresponding test probe to provide a measure of the signal due to specific binding of the test probe.

The concentration of a particular sequence can then be determined by measuring the signal intensity of each of the probes that hybridize selectively to that gene and normalizing to the normalization controls. Where the signal from the probes is greater than the mismatch, the mismatch is subtracted. Where the mismatch intensity is equal to or greater than its corresponding test probe, the signal is ignored. The expression level of a particular gene can then be scored by the number of positive signals (either absolute or above a threshold value), the intensity of the positive signals (either absolute or above a selected threshold value), or a combination of both metrics (e.g., a weighted average).

In certain embodiments, normalization controls are often unnecessary for useful quantification of a hybridization signal. Thus, where optimal probes have been identified, the average hybridization signal produced by the selected optimal probes provides a good quantified measure of the concentration of hybridized nucleic acid.

Where mismatch controls are present, the detecting step may comprise calculating the difference in hybridization signal intensity between each of the oligonucleotide probes and its corresponding mismatch control probe. The detection step may further comprise calculating the average difference in hybridization signal intensity between each of the oligonucleotide probes and its corresponding mismatch control probe for each gene.

In applications of the invention whereby two or more resolvable labels are combined on the same array, the ratio of the signals from each separately labeled target may be calculated and used to define relative levels of expression or representation.

Splice Junction Index, Intron Accumulation Index and Precursor/Mature Index

Hybridization signals from two different samples co-hybridized to a single array are quantified and processed. The ratios from separate array elements targeted to different parts of the same gene are then related to each other to create “indexes” that describe the changes in relative amount of different targets in the different samples. In preferred embodiments, splicing is expressed using a Splice Junction (SJ) Index, an Intron Accumulation (AI) Index or a Precursor/Mature (PM) Index, which indices represent the ratio of the normalized ratio of a particular splicing product in two nucleic acid samples. The SJ, AI and PM Indices are described below.

The SJ index is the ratio of normalized signals generated from test nucleic acid samples co-hybridized against the same array where the nucleic acid samples are each differently labeled (e.g. with Cy3 or Cy5). The SJ index may be expressed using the following formula:
SJ1/SJ2 divided by E1/E2,

where SJ1 and SJ2 represents the quantity of hybridization to a particular splice junction probe in nucleic acid samples 1 and 2, respectively and E1 and E2 represents the quantity of hybridization to a particular exon probe, which may be a probe for a constitutively spliced exon, in nucleic acid samples 1 and 2, respectively. In a preferred embodiment, the SJ index is obtained by converting the SJ1/SJ2 and E1/E2 ratios into log₂ ratios, and SJ Index is calculated by subtracting the log₂ ratio of the intron probe from the log₂ ratio of the splice junction probe.

An IA Index is obtained in a similar manner to the SJ Index, except intron probe hybridization signal levels are used in the formula instead of a splice junction probe hybridization signal levels. The IA Index is preferably calculated by subtracting the log₂ ratio of the intron probe from the log₂ ratio of the exon probe.

A PM index is obtained in a similar manner to the SJ Index, except intron probe hybridization signal levels were used in the formula instead of exon probe hybridization signal levels. The PM Index is preferably calculated by subtracting the log₂ ratio of the splice junction probe from the log₂ ratio of the intron probe. This index mimics the unspliced/spliced ratio used in classical splicing studies (C. W. Pikielny, M. Rosbash, Cell 41, 119-26 (1985)).

The SJ, IA, and PM indices are useful alone or in combination to, for example, compare relative exon usage and/or and ratios of various mRNA splice variants under various conditions. For example, the effect of certain mutations in genes implicated in the splicing apparatus can be assessed. In another example, the indices can be used to analyze the effects of agents (e.g., candidate drugs, drugs of known activity, endogenous factors, and the like) upon splicing of one or more selected genes. In another example, splicing changes of normal developmental processes, disease processes and physiological responses to changes in the environment can be monitored.

Kits

Also provided are kits for performing assays using the subject devices, where kits for carrying out differential splicing analysis assays are preferred. Such kits according to the subject invention will at least comprise the subject arrays. The kits may further comprise one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like.

Specific Arrays of the Invention

In certain embodiments, the array of the kit is of a specific type in that all of the probes on the array are for detection of alternative splicing of one or more genes, which genes may be of the same type or origin, or have some other shared feature, as discussed above in detail. A variety of specific array types are contemplated, including, but not limited to: human, cancer, apoptosis, mouse, stress, oncogene, tumor suppressor, cell-cell interaction, cytokine and cytokine receptor, rat, rat stress, blood, neuroarray, and the like.

In general, the number of genes represented on an array is at least 2, and can be 5 or more, 10 or more, 20 or more, 50 or more, 100 or more, up to the limits of the surface area of the substrate and the ability of the detection methods to distinguish detectable signals from the spots of on the array. In one embodiment, probes present on the array provide for detection of at least two mRNA splice variants of the same gene, and may represent 3 or more such mRNA splice variants, and may provide for detection of all mRNA splice variants of each gene represented on the array.

Utilities of the Invention

The subject compositions and methods find use in, among other applications, splicing assays. As such, one may use the subject methods in the analysis of various nucleic acid samples that are suspected of containing spliced products. The following utilities are or particular interest: (a) analysis of the rate of splicing for a particular intron; (b) profiling of mRNA splicing of a selected gene in cells that differ in disease (in particular cancer) states, developmental stage, tissue origin, exposure to environmental factors, and the like; (c) analyzing of the protein variants produced by a selected gene in cells that differ in disease (in particular cancer) states, developmental stage, tissue origin, exposure to environmental factors, and the like; and (d) identifying and validating of candidate splice junctions.

In one embodiment, the arrays and assays of the invention are used in the identification and analysis of factors that modulate splicing (e.g., increase or decrease splicing rate, e.g., with respect to a given splice variant, including regulation of splicing). Exemplary factors that can be analyzed for their effect upon splicing include, but are not necessarily limited to, genetic factors (endogenous to a cell or exogenously introduced), agents (e.g., candidate drugs, known drugs, and the like), and environmental factors (e.g., stress, chemical factors, osmolarity, temperature, and the like).

For identification of factors that regulate splicing, naturally occurring and engineered variants or mutants encoding mRNA splicing machinery may be utilized to elucidate the role of a particular polypeptide in splicing and/or modulation of splicing by the factor being analyzed. For example, in the analysis of genetic factors, the data provided by assays using the arrays of the invention may be processed using supervised or unsupervised clustal analysis (e.g. Brown et al., Proc. Natl. Acad. Sci. 2000 97:262-267) and relationships between splice sites, splicing factors and biological functions can be built. Using these methods, a particular splicing factor can be linked to splicing of a particular gene or group of genes involved in a particular biological function. Once a particular splicing factor has been identified, the sequence that the splicing factor binds to can be elucidated using biochemical or using bioinformatics approaches, and small molecules, in particular antisense and binding site mimetics can be developed to specifically inhibit the activity of the splicing factor. Using this method a drug that influences the splicing of a particular gene can be developed, for example a therapeutic nucleic acid that can induce apoptotic splice variants of bcl-x can be developed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

To discriminate between spliced and unspliced RNAs for each intron-containing yeast gene, we used DNA microarrays (J. L. DeRisi, V. R. Iyer, P. O. Brown, Science 278, 680-6. (1997); D. J. Lockhart, E. A. Winzeler, Nature 405, 827-36. (2000)). Oligonucleotides were designed to detect the splice junction (specific to spliced RNA and not found in the genome), the intron (present in unspliced RNA), and a second exon (common to spliced and unspliced RNA) for each intron-containing gene as shown in FIG. 1A. The oligos were printed on glass slides to create splicing-sensitive microarrays for yeast.

Pilot experiments indicated that 40 nt provided a reasonable compromise between strength of signal and specificity under the hybridization conditions of ˜0.78 M Na+ and 62° C., at 21-24° C. below the predicted Tms (J. SantaLucia et al. Biochemistry 35, 3555-62. 1996). The splice junction oligonucleotides were designed with the exon-exon junction positioned between residues 20 and 21 of the 40 mer. This set of oligos has predicted Tms distributed around 84.6±3.5° C., at 1 M Na+. We chose the remaining intron and exon oligos based on four criteria: (a) predicted Tm near 84.6° C., (b) limited internal secondary structure, (c) a modification of aheuristic that discourages low information content (D. J. Lockhart et al., Nat Biotechnol. 14, 1675-80. 1996), and (d) absence of significant homology with more than one place in the yeast genome as determined by BLAST. A set of 832 40-mers with 5′ amino linker modifications was custom ordered through an informal agreement with EOS Biotechnology (http://www.eosbiotech.com). Oligonucleotides (70-mers with 5′ amino linkers) for all yeast genes were purchased as a set from Operon, Inc. These are designed to have Tms of 74±3° C. in 0.1M Na+, which translates to 86.5±3° C. in 1M Na+, comparable to the estimated Tms of the set of 40-mers we designed.

Oligos with 5′ amine linkers were printed onto glass slides at a concentration of 10 pmol/ul in 150 mM Sodium Phosphate (pH 8.5) using a robot built according to specifications from J. DeRisi posted on a website supported by Pat Brown's laboratory at Stanford University, Center for Molecular and Genetic Medicine. In slide format 1, each 40-mer was printed in quadruplicate, resulting in microarrays that contain more than 3400 elements and four fold oversampling. Slide format 2 contained four copies of the set of 40-mers plus 70-mers for about a thousand intronless genes. Format 3 contains two copies of 40-mers plus the complete Operon set. Slides were purchased from SurModics, Inc. (now available from Motorola, Inc.) and are coated with a three-dimensional polymer that covalently binds free amines. After printing, slides are incubated in a humid chamber for 36-48 hours. Prior to use residual reactive groups are blocked wit 50 mM ethanolamine in 0.1M Tris (pH=9.0)/0.1% SDS at 50° C. for 30 mins. Slides are then washed with 4×SSC/0.1% SDS at 50° C. for 15 min, rinsed with water and spun dry. To determine whether oligonucleotide arrays can function as genome-wide sensors of splicing, we compared RNA of cells carrying the temperature sensitive splicing mutation prp4-1 with RNA of wild type (wt) during a shift from 26° C. to 37° C. Prp4p is an integral component of the spliceosome (J. Banroques, J. N. Abelson, Mol Cell Biol 9, 3710-9. (1989); S. P. Bjorn, A. Soltyk, J. D. Beggs, J. D. Friesen, Mol Cell Biol 9, 3698-709 (1989)) FIG. 1B shows plots of fluorescence for each oligo for the wild type (Cy3) versus the prp4-1 mutant (Cy5) with time. Fluorescently labeled target sequence sample preparation and hybridization were performed as described (J. L. DeRisi, V. R. Iyer, P. O. Brown, Science 278, 680-6. (1997)) using 20 ug of total RNA primed with a mixture of oligo dT and random hexamers. Arrays were scanned and analyzed using a GenePix 4000A scanner and GenePix Pro 3.0 software from Axon Instruments (Union City, Calif.).

Even at the permissive temperature of 26° C., many intron probes (red spots) display Cy5/Cy3 ratios >1, indicating accumulation of intron-containing RNA in the mutant strain. After the shift to restrictive temperature, the Cy5/Cy3 ratio increases for most intron probes. In contrast, the ratio decreases for many splice junction probes (green spots), a sign that spliced RNAs become depleted in the mutant. The Cy5/Cy3 ratios for about a thousand intronless genes remain largely unaffected (yellow spots). Thus the array reports a catastrophic splicing defect and can measure the kinetics of splicing inhibition genome-wide.

### Example 2

Despite their conservation, numerous mRNA processing factors are not essential in yeast. To analyze more subtle changes in splicing, we studied 18 mutant strains lacking nonessential genes implicated in mRNA processing (Table 1).

TABLE 1
mRNA processing genes used in this study.

| Gene | ORF | Product |
|---|---|---|
| GCR3, STO1, CBC1, | YMR125w | nuclear cap binding |
| CBC80 | | complex subunit |
| MUD13, CBC2, CBC20 | YPL178w | nuclear cap binding |
| | | complex subunit |
| NAM8, MRE2, MUD15 | YHR086w | U1 snRNP protein |
| MUD1 | YBR119w | U1 snRNP A protein |
| MUD2 | YKL074c | commitment complex protein |
| MSL1, YIB1 | YIR009w | U2 snRNP B″ protein |
| CUS2 | YNL286w | U2 snRNP protein |
| SNU17, IST3 | YIR005w | U2 snRNP protein |
| PRP4 | YPR178w | U4/U6 snRNP protein |
| SNU40 | YHR156c | U5 associated |
| SNU66 | YOR308c | U4/U6.U5 tri-snRNP |
| ECM2, SLT11 | YBR065c | U2/U6 associated, 2nd step |
| PRP18 | YGR006w | U5 snRNP protein, 2nd step |
| PRP17 | YDR364c | 2nd step |
| BRR1 | YPR057w | snRNP biogenesis/recycling |
| UPF3 | YGR072w | nonsense mediated decay |
| DBR1, PRP26 | YKL149c | debranching enzyme |
| HSP104 | YLL026w | splicing and heat shock |

All strains used except prp4-1 and its wild type reference were derived from BY4741. All genes are nonessential except PRP4. Additional information concerning these genes is available at the Stanford Genome Database website, which website is supported by Stanford University.

FIG. 1C shows plots of mutant versus wild type fluorescence intensities for prp18Δ, cus2Δ, and dbr1Δ. The effect of each deletion on spliced and unspliced RNA is different. Most severe is prp18Δ, which causes widespread intron accumulation and loss of splice junction sequences relative to wild type (FIG. 1C, left). The cus2Δ mutation enhances defects in U2 snRNA or Prp5p (D. Yan et al., Mol Cell Biol 18, 5000-9 (1998); R. Perriman, M. Ares, Jr., Genes Dev 14, 97-107. (2000)), but causes little intron accumulation (FIG. 1C, center). Although not required for splicing, Dbr1p debranches the lariat, and its loss results in the dramatic accumulation of intron lariats (K. B. Chapman, J. D. Boeke, Cell 65, 483-92 (1991)). In the dbr1Δ strain most introns accumulate, and there is little effect on spliced mRNAs (FIG. 1C, right). This demonstrates that subtle qualitative differences in splicing phenotype can be distinguished using splicing sensitive microarrays.

### Example 3

Changes in spliced and unspliced RNA levels due to loss of an mRNA processing factor may arise directly from splicing inhibition or may be due to secondary events that alter transcription or RNA decay. For example, signal from a splice junction probe may increase for a gene whose transcription is induced, even though splicing is inhibited. To account for such effects, we devised two gene-specific indexes that relate changes in spliced and unspliced RNA to changes in total transcript level. The splice junction index (SJ) relates gain (or loss) of splice junction probe signal to gain (or loss) of total gene-derived signal as measured by the corresponding exon 2 probe. Similarly, the intron accumulation (IA) index relates changes in signal from the intron probe to its corresponding exon 2 probe.

In the present example, the Splice Junction (SJ) Index is the ratio of the mutant/wild type ratios derived from the normalized signals from the splice junction probe to the exon2 probe: SJ Index=SJmut/SJwt divided by E2mut/E2 wt, obtained by subtracting the log2ratio of the exon2 probe from the log2ratio of the splice junction probe. The Intron Accumulation (IA) Index is obtained by subtracting the log2ratio of the exon2 probe from the log2ratio of the intron probe. Because probe performance may not be directly related to absolute transcript amount, these indexes depend idiosyncratically on the sequences of the probes. We also calculated the precursor/mature (PM) index, which is obtained by subtracting the log2ratio of the splice junction probe from the log2ratio of the intron probe. This index mimics the unspliced/spliced ratio used in classical splicing studies (C. W. Pikielny, M. Rosbash, Cell 41, 119-26 (1985)).

To measure their splicing differences with more statistical confidence we averaged fluor-reversed pairs of experiments for each mutant to remove labeling bias (J. DeRisi et al. Nature Genetics 14:457-60. 1996). Normalization of the data was accomplished by the method of Chen (Y. Chen et al., J. Biomed. Optics 2:364-374. 1997) using “Norm” a custom written software application. Norm also screens out artificially high ratios on spots of low signal by adding a prior of two standard deviations of the background to both channels before calculating the ratio. Norm allows users to select control spots to use for nomalization and automatically flags spots with low intensity, high background, or high saturation. The signals from the coding regions of eight intronless genes expressed at unchanging levels in 80 yeast microarray experiments from Pat Brown's lab (the stoic genes) were used for normalization. These stoic genes (SLY1, YDR189w, vesicle trafficking between ER and Golgi; SEC4, YFL005w, Golgi to plasma membrane transport; VPS45, YGL095c, Golgi vacuole transport; PEX4, YGR133w, Ubiquitin conjugating enzyme; TAF145, YGR274c, RNA Pol II general transcription factor; APS3, YJL024c, transport of alkaline phosphatase to the vacuole; RSC2, YLR357w, chromatin remodeling; YAP1, YML007w, jun-like transcription factor) fall into different functional classes other than mRNA processing and display a broad range of expression levels. Experiments with whole genome arrays show that normalization factors derived from these stoic genes are equivalent to the normalization factors obtained using all intronless genes in the genome.

Log2ratios from the four replicate array elements for each probe and reverse labeled experiments (typically a total of eight measurements) were averaged for each probe and clustered using software written by M. Eisen (available on the internet through the company microarrays.org). Reproducibility and noise estimates were made using six different array batches, four independent wild type by wild type comparisons, and multiple cultures of the same mutant strains compared to wild type. Intron signals for some genes expressed at low levels in wild type cells are undetectable I some cases, but become detectable upon splicing inhibition or I the dbr1Δ mutant. Intron-containing transcripts from genes expressed at high levels are readily detected in growing wild type cells, as expected if a few percent of transcripts from each gene are yet to be spliced. Clustering in which the wild type by wild type experiments are included with the data presented here show that the hsp104Δ mutant is indistinguishable from wild type

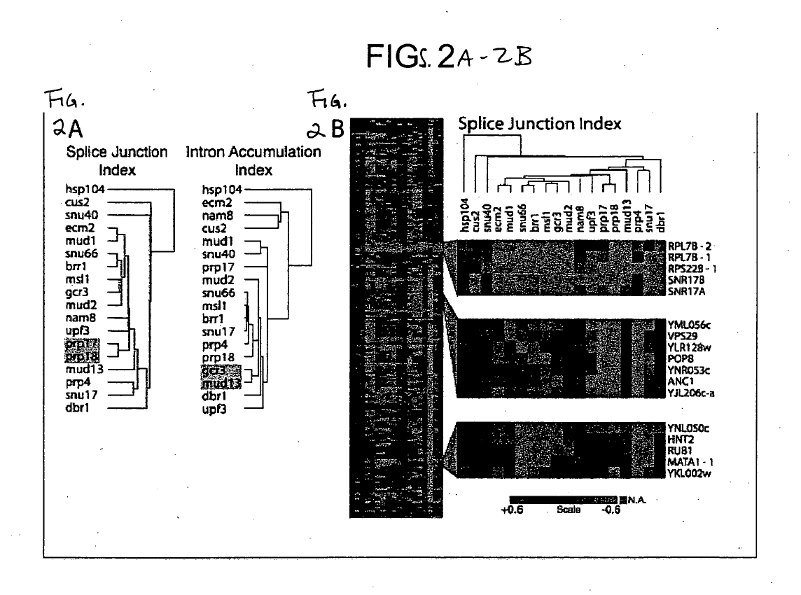

We calculated both indexes for each intron-containing gene, clustered the indexes, and compared the relationships of the mutant strains revealed by their genome-wide splicing phenotypes (FIG. 2A).

A striking conclusion from this comparison is that different mutations have distinct effects on spliced (SJ Index cluster) and unspliced (IA index cluster) RNA. This means that the SJ index detects a different set of consequences of mRNA processing factor loss than the IA index. Furthermore, there appears to be no general formula to describe the relationship between the loss of spliced RNA and the accumulation of unspliced RNA. Early studies assumed a simple relationship between these processes (C. W. Pikielny, M. Rosbash, Cell 41, 119-26 (1985)) and have used the change in ratio of unspliced to spliced RNA or the increase in unspliced RNA to the total as a measure of splicing inhibition. This indicates that information may be gleaned by considering the indexes separately (FIG. 2A).

### Example 4

To test this we examined the clusters in light of known functional relationships between mRNA processing factors. The IA indexes derived from loss of the two subunits of the nuclear cap binding complex Mud13p and Gcr3p (H. V. Colot, F. Stutz, M. Rosbash, Genes Dev 10, 1699-708 (1996); P. Fortes et al., Mol Cell Biol 19, 6543-6553 (1999)) cluster together (r=0.88), whereas their SJ indexes do not. This indicates that the genome-wide effect of their loss on intron accumulation is much more similar than their effect on splice junctions, and is distinct from the effects of other mutations on intron accumulation (FIG. 2A). This could be due to a function of the complete nuclear cap-binding complex specific to intron-containing RNA. The failure of mud13Δ and gcr3Δ SJ indexes to cluster may be explained if one subunit has a partial function specific to spliced RNA that does not require the other subunit (E. C. Shen, T. Stage-Zimmermann, P. Chui, P. A. Silver, J Biol Chem 275,23718-24. (2000)). Also notable is the dissimilarity in the intron accumulation patterns of mutants lacking Prp17p and Prp18p, in contrast to their much more similar effects on splice junction levels (r=0.82). This implies that the fate of incompletely spliced transcripts is different in these mutants, despite the expectation (supported by the SJ index) that they work together at or near the same step in splicing (M. H. Jones, D. N. Frank, C. Guthrie, Proc Natl Acad Sci USA 92, 9687-91. (1995).).

We next asked whether intron-containing genes depend on mRNA processing factors to different extents. The genome-wide response to loss of individual factors is complex, suggesting a variety of dependencies (FIG. 2B, left). The top panel (FIG. 2B, right) shows a group of genes that appear to be affected by the loss of most nonessential factors. The middle panel shows a small cluster of genes that are primarily affected by the loss of Prp17p and Prp18p, but not greatly affected by the loss of other factors. The bottom panel shows a group whose splicing is weakly affected by loss of Prp17p and Prp18p, but more severely decreased in strains lacking Snu66p, Brr1p, and Msl1p. Each intron-containing gene shares a distinct set of factor dependencies for RNA splicing with a relatively small number of other genes. These dependencies also do not align in snRNP-specific fashions, since patterns produced by loss of Mud1p and Nam8p, both U1 snRNP proteins, are distinct from each other, as are those of the U2 snRNP proteins Ecm2p, Cus2p, and Msl1p. In contrast, Mud1p and Ecm2p produce similar patterns (r=0.83) suggesting a cooperative function between a U1 and a U2 snRNP protein.

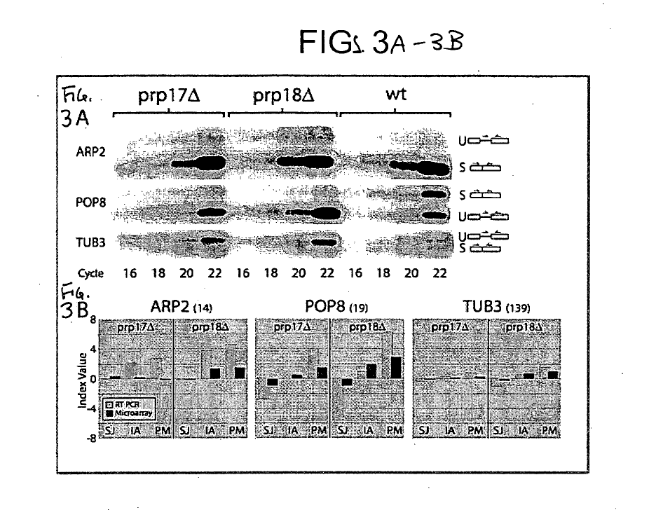

To test the robustness of an array-based observation, we validated a small fraction of the array data relevant to a prevailing hypothesis for Prp18p function using RT-PCR (FIG. 3). Based on splicing of mutant ACT1 reporter substrates in vitro, Prp18p is hypothesized to be dispensable for splicing when the branchpoint (bp) to 3′ splice site (ss) distance is ≦17 nt, and is increasingly required in vitro as this distance increases (Zhang and Schwer (Nucleic Acids Res 25, 2146-52 (1997)) define the bp to 3′ ss distance starting from 2 bases downstream of the bp adenosine to the Y of the 3′ ss YAG sequence. Therefore, their distance of 12 nt corresponds to 17 nt from the actual branched A to the 3′ splice site G). A comparison of bp to 3′ ss distances with either SJ or IA index values from prp18Δ experiments for natural introns shows no correlation. Since prp18Δ clusters with prp17Δ, we included both for validation (FIG. 3B). Some genes with short bp to 3′ ss distances are relatively unaffected by loss of Prp17p and Prp18p (e.g. RUB1, 12 nt, FIG. 2B bottom right panel, PCR data not shown). However, two introns with short distances are detectably affected (FIG. 3B). POP8, with a bp to 3′ ss distance of only 19 nt, was the intron most affected by loss of Prp18p (FIG. 3B). Conversely, several introns with long bp to 3′ ss distances are not drastically affected. TUB3, containing the intron with the largest distance (139 nt) is only weakly affected (FIG. 3B). With respect to the genes we tested the two kinds of data provide the same trends (FIG. 3B). This confirms changes in splicing detected by the array, and suggests that hypotheses concerning mRNA processing factor function can be refined using this approach.

To test this, we evaluated additional hypotheses concerning mRNA processing factor function in light of the array data. We find that the expectation that nonsense-mediated decay is generally important for reducing the levels of unspliced RNA in the cytoplasm (F. He, S. W. Peltz, J. L. Donahue, M. Rosbash, A. Jacobson, Proc Natl Acad Sci USA 90, 7034-8 (1993); M. J. Lelivelt, M. R. Culbertson, Mol Cell Biol 19, 6710-9. (1999)) is not supported by the observation that the majority of these do not accumulate significantly in a upf3Δ strain. The expectation based on intronic snoRNA processing phenotypes that accumulation of introns in the dbr1Δ mutant should be inversely related to intron size (S. L. Ooi, D. A. Samarsky, M. J. Fournier, J. D. Boeke, Rna 4, 1096-110. (1998)) seems not to hold either, most likely due to Dbr1p-independent mechanisms of intron turnover. We do not observe correlation between a nonconsensus 5′ splice site or a U-rich region near the 5′ splice site and strong dependence on Nam8p (O. Puig, A. Gottschalk, P. Fabrizio, B. Seraphin, Genes Dev 13, 569-80 (1999)) for splicing in vivo. We also see no correlation between the presence of a U residue upstream of the branchpoint sequence (J. C. Rain, P. Legrain, Embo J 16, 1759-71 (1997)) or the presence of a polypyrimidine tract before or after the branchpoint and strong dependence on Mud2p. These data indicate that using any one intron as a reporter may cause the importance of a factor to be overemphasized or missed. Genome-wide analysis allows perturbations of splicing to be evaluated on every intron at once, in effect using the entire genome as a reporter.

### Example 5

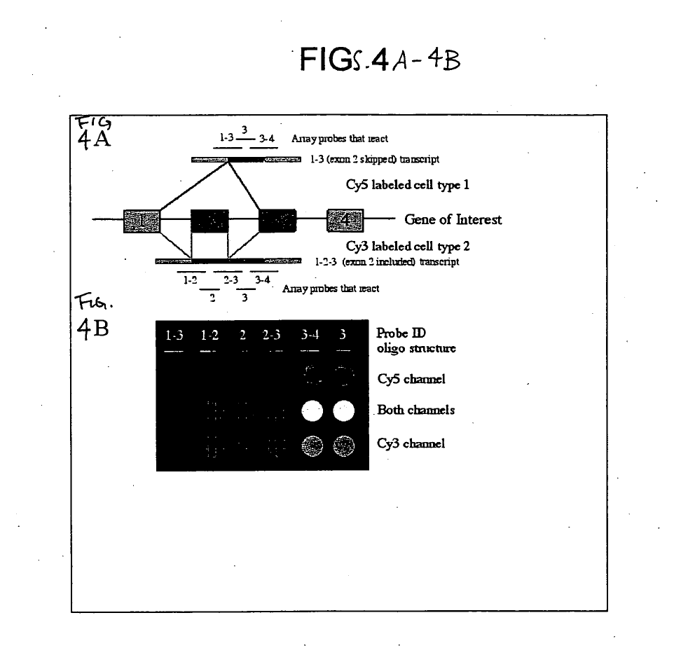

The strategy we use to discern alternative patterns of splicing using a microarray format is shown in FIG. 4. Rather than use large PCR products or oligos each representing a gene, we identify the key regions in which different mRNA isoforms from the same gene differ from each other and use short oligos designed to be specific for these differences. In the example shown, a gene produces two variant mRNAs that differ by the skipping or inclusion of exon 2 (FIG. 4A). In cell type 1, exon 2 is skipped, and in cell type 2, it is included. Idealized data envisioned by conceptually labeling cell type 1 mRNA sequences with Cy5 and cell type 2 mRNA with Cy3, mixing and hybridizing to an array of oligonucleotides specific for the exon1-exon2 splice junction (1-2), the exon1-exon3 junction (1-3), internal exon2 sequences (2), etc is shown in FIG. 4B. From the Cy5/Cy3 ratios obtained, we can deduce both the relative levels of mRNA in each cell type using features common to both isoforms (e.g. 3, or 3-4), as well as determine the relative levels of each isoform in the pool of mRNA derived from the gene. This approach results in log₂ ratios for each feature, as well as indexes (ratios of log₂ ratios) that address alternative splicing for each gene.

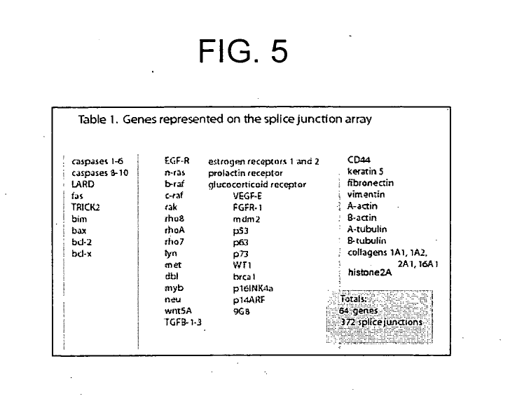

We selected for study numerous genes implicated in growth control, cancer, or apoptosis and for which there was some evidence in the literature that alternative splicing might regulate the activity of the gene product (FIG. 5). Some genes have been selected to allow us to compare our array data with published data from other labs. Other selected genes ensure that we have transcripts commonly found in all cell types as standard indicators of experimental quality and data normalization.

Once genes are identified, the regions of the transcript that differ in the different isoforms are identified. A major tool in this analysis is the UCSC Human Genome Browser, which presents alignments of available cDNA sequences with the draft genome sequence. Yeast studies show that 40-mers symmetrically spanning the splice junction provide a reasonable compromise between the competing considerations of signal strength, cross-reaction and specificity for the intended target. With limited resources, we are unable to explore optimization in any depth for any one junction sequence, however we have criteria for selecting oligonucleotides for exon sequences where we have more flexibility. Sequences are evaluated for secondary structure, and melting temperature. We use automated BLAST and a faster algorithm called to screen out candidate sequences with spurious complementarity to unintended targets. The array has 372 human splice junction features representing thousands of alternatively spliced isoforms. In total, 484 40-mer oligonucleotides were designed from cDNA alignments to the human genome. Four spots of each oligonucleotide are printed on each slide to allow for four-fold oversampling.

### Example 6

An initial series of experiments comparing HeLa cells and 293 cells were performed. RNA was extracted, labeled using a random primer and reverse transcriptase, hybridized to the arrays and log₂ ratios for each oligo were compared after averaging fluor-reversed duplicate arrays, typically resulting in 8 measurements per oligonucleotide. Each oligonucleotide likely has a different efficiency of hybridization, and different regions of the mRNA may be more or less well represented in the labeled cDNA. Data from control genes that do not display alternative splicing indicates that the expression of these genes (actins, tubulins, histone, GAPDH) in the two cell lines is only slightly different (less than 2-fold). The spot to spot standard deviations are reasonable for most of the features, demonstrating that the signals are uniform. The variation between features targeting the same control mRNA is also reasonably good. Low feature to feature variation within constitutively spliced mRNA regions is important in order to distinguish alternatively spliced regions.

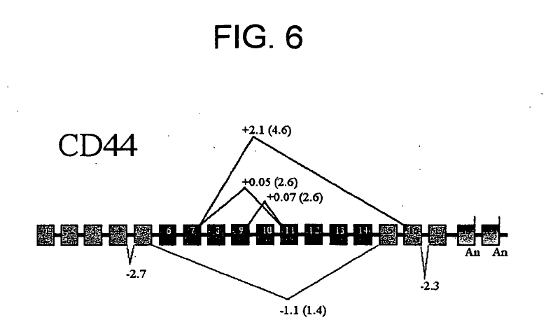

To determine whether we observe alternative splicing we analyzed data for CD44 (FIG. 6). In this case we compared the ratios for two constitutive splicing events (exons 4-5 joining and 16-17 joining) with other splicing events involving the cassette exons. The two constitutive splice junction oligos suggest that the log ratio that best describes the relative levels of all CD44 mRNA isoforms in the two cell lines is −2.5, indicating that the total CD44 mRNA levels are about 5.7 fold higher in 293 than HeLa. (The 293 cells are adherent, however the HeLa cells are grown in spinner bottles). In the absence of any alternative splicing differences in the two cell lines, every CD44 feature should give about the same log₂ ratio, as observed in the control genes. However, other junctions differ substantially, indicating alternative splicing. The most dramatic example is the 7-216 junction, which is 4.3-fold more common in HeLa cells than 293 cells. To illustrate the relative representation of this junction in the CD44 mRNA pool, we normalize to the constitutive CD44 measurements to create a specific index. To obtain the index in log space we subtract the log₂ ratio of the constitutive splice junction(s) from that of the alternative junction. The indexes so derived are shown in parentheses in FIG. 2. An index of 4.6 for the 7-16 junction indicates that it is 24-fold more common in the CD44 mRNA pool of HeLa than 293 cells. It is important to consider both numbers, since fluctuation in absolute mRNA isoform level per cell, and fraction of gene-derived mRNA as a particular isoform are relevant. We conclude that our approach is viable for profiling alternative splicing patterns in mammalian cells.

These studies present the first genome-wide view of splicing for any organism. The ability to distinguish differently spliced forms of RNA using oligonucleotide microarrays opens the way for expression profiling that accounts for alternative splicing and splicing regulation in higher cells. Estimates suggest that 40-60% of human genes produce alternatively spliced transcripts (E. S. Lander et al., Nature 409, 860-921. (2001); B. Modrek, C. Lee, Nat Genet 30, 13-9. (2002)). In a growing number of key cases, alternatively spliced mRNAs produce proteins of distinct or even antagonistic function (e.g. (L. H. Boise et al., Cell 74, 597-608. (1993)). Improved expression profiling technologies must resolve changes in alternative splicing not simply by estimating exon representation (e.g. (D. D. Shoemaker et al., Nature 409, 922-927 (2001)), but by providing direct evidence for exon joining. The results we describe here demonstrate that oligonucleotide arrays designed to detect specific splicing products will be key to accurate parallel analysis of alternative splicing in higher organisms.

It is evident from the above results and discussion that the subject invention provides an important new means for investigating splicing. Specifically, the subject invention provides a system for analyzing, in parallel the expression of several splice variants of several genes. As such, the subject methods and systems find use in a variety of different applications, including research, proteomics, drug discovery, profiling and other applications. Accordingly, the present invention represents a significant contribution to the art.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1.-21. (canceled)

22. A method of analyzing mRNA splice products of a selected gene, the method comprising:
contacting a first nucleic acid sample with a nucleic acid array comprising:
splice junction probe that specifically hybridizes to a non-genomic sequence spanning a splice junction between a first exon and a second exon in an alternatively spliced mRNA gene product of the selected gene; and
a control probe that specifically hybridizes to a splice junction of a constitutively spliced mRNA gene product of the selected gene;
wherein said contacting is under conditions to provide for specific hybridization of the first nucleic acid sample to the splice junction probe and the control probe; and
detecting a first splice junction probe signal and a first control probe signal for the first nucleic acid sample;
contacting a second nucleic acid sample with an array comprising the splice junction probe and the internal control probe of the array contacted with the first nucleic acid sample, wherein said contacting is under conditions to provide for specific hybridization of the second nucleic acid sample to the splice junction probe and the control probe;
detecting a second splice junction probe signal and a second control probe signal for the second nucleic acid sample;
comparing a first ratio of first and second splice junction probe signals to a second ratio of the first and second control probe signals;
wherein a difference in the first and second ratios is indicative of the presence of alternatively spliced gene product in the first and second nucleic acid samples.

23. The method of claim 22, wherein
the first ratio is a log2 ratio of the first splice junction probe signal and the second splice junction probe signal; and
the second ratio is a log2 ratio of the first control probe signal and the second control probe signal.

24. The method of claim 22, wherein the array comprises more than on control probe, which control probes hybridize to different splice junctions of a constitutively spliced gene mRNA product, and wherein the second ratio is an average of ratios of first and second control probe signals from the control probes.

25. The method of claim 22, wherein
the array comprises more than on control probe, which control probes hybridize to different splice junctions of a constitutively spliced gene mRNA product;
the first ratio is a log2 ratio of the first splice junction probe signal and the second splice junction probe signal; and
the second ratio is an average of log2 ratios of the first and second control probe signals from the control probes.

26. The method of claim 22, wherein the first nucleic acid sample is labeled with a first label, and the second nucleic acid sample is labeled with a second label, wherein said first and second labels are different, and wherein the first and second nucleic acid samples are contacted with the same array.
